(19) United States
(12) Patent Application Publication
(10) Pub. No.: US 2008/0243088 A1
(43) Pub. Date: Oct. 2, 2008

(54) Radio frequency identification drug delivery device and monitoring system

(75) Inventor: Robert F. Evans, Mobile, AL (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: DocuSys, Inc., Mobile, AL (US)

(21) Appl. No.: 11/729,995

(22) Filed: Mar. 28, 2007

(51) Int. Cl. A61M 5/00

(52) U.S. Cl. 604/246; 222/30

(57) ABSTRACT

A drug delivery device, system and method for identifying a drug contained in the drug delivery device and the amount of the drug administered. The drug delivery device includes at least a first portion and a second portion, in which the portions move relative to one another as a drug contained in the drug delivery device is administered or expelled from the drug delivery device. The drug delivery device includes radio frequency identification (RFID) tags that are mounted on each of the portions. At least one active tag is mounted on one of the first and second portions.

### BACKGROUND OF THE INVENTION

The present invention relates in general to the field of drug delivery, and more particularly, to syringes and similar devices for delivery of medicament or drugs to medical patients.

Adverse drug events that lead to injury or death are a major concern in the health care industry. Often, such events include administration of the incorrect dosage or wrong drug to a patient. In view of these problems, devices and methods for monitoring the amount of a drug administered and for identifying characteristics of the drug to be administered have been developed.

One known method for determining real-time administration dosages involves the use of optical technology to determine the location of a plunger within a syringe. In a syringe, depression of the plunger causes medication within the syringe to be dispensed into the patient. In optical technology, digital images of the syringe are captured. Through image processing, the end of the plunger is located in relation to a fiducial marker on a syringe label cradle (SLC), which acts as a holder and positioner for the syringe. As the plunger is depressed, the capture and processing of sequential images of the syringe enable tracking of the plunger as it advances in the syringe. See U.S. Pat. Nos. 5,651,775 and 6,885,678, which disclose optical systems for determining the content volume in a syringe.

Other methods of determining drug volume in a syringe include the use of inductive and capacitance methods. These methods generally involve applying a voltage or magnetic field to the syringe, which generates an electrical response. Once repeated, the change in a parameter, either voltage or capacitance, reveals the amount of a drug administered. See U.S. Pat. Nos. 5,720,733, 6,068,615, 6,110,148, 6,113,578 and 6,352,523, which describe inductive and capacitance methods for determining drug volume in a syringe.

For identification of the contents of a syringe, one known technique is the use of bar codes. In bar code technology, when a syringe is filled with a drug, a bar code identifying the drug is affixed onto the syringe. When the syringe is passed through a bar code reader, the contents of the syringe are identified. For example, see U.S. Pat. No. 5,383,858, which discloses bar coded syringes for use with a syringe pump.

Instead of the above-mentioned methods of monitoring real-time dosage amounts and identifying the contents of a drug delivery device, the present method employs radio frequency identification (RFID) for these purposes.

RFID has gained prominence as a means of equipment and product supply-line tracking as well as a means to track other subjects, including personnel and even children at theme parks. One great advantage of this technology is its ability to specifically identify individual items or people without requiring individual scanning of each separate entity. For example, a whole carton or packing case of products can be audited or detected without opening a shipping container holding those products.

Generally, RFID is an identification method that utilizes devices (e.g., RFID tags or sensors) to store and remotely retrieve data. These devices are able to store and transmit radio waves to other such devices or to antennae, or other types of sensing devices.

RFID systems may include the use of either or both active and passive technologies. In active RFID technology, an “active device” broadcasts its existence and identifier to a receiving antenna. These devices are self-powered, with an internal energy source, such as a battery. In passive RFID technology, “passive devices” are capable of receiving a radio pulse at one frequency, storing the energy in a capacitor and then, retransmitting a second pulse. In this manner, passive devices do not have an inherent power source. Instead, passive devices typically depend on a monitoring source signal for activation. This activation usually involves receipt of a radio pulse at one frequency and storing the energy from the frequency in a capacitor. Once activated, a passive device re-transmits the signal or disturbance or absorbance of the signal (usually at a different frequency) to signify the presence of the passive device.

Both active and passive technologies are extensively used in many industries. For example, active and passive technologies have been used to monitor specific portals, such as the exits of retail stores, for items leaving a premise. In this capacity, the devices may be used to prevent theft or shoplifting, as part of a library book check-out system, or as video rental monitoring equipment. For example, a typical anti-shoplifting tag or label is a passive device. If an anti-shoplifting tag has not been deactivated by a shopkeeper or store clerk, the tag will retransmit a radio wave it receives when a shopper carrying an item with the tag passes through a sensing portal placed at a store exit. Meanwhile, active devices are popular in hospitals and other large organizations or companies to track staff or equipment. The staff or equipment carry active sensors that continually transmit their presence to specially designed receiving antennae strategically located throughout a facility. This allows the staff or equipment to be tracked and for a centralized display of the locations of staff or equipment.

The use of RFID technology has advantages over those technologies currently used to identify a syringe's contents or to monitor the dosage administered. For example, bar code technology allows for the tracking of drug delivery devices, such as syringes, but each device must be passed through or next to a scanner in order for the drug information on the bar label to be read or inputted into a computer. RFID technology, on the other hand, allows for the delivery of information from a source to a recipient while the source is at a location remote from the recipient. Unlike the relationship between a bar code label and a scanner, an RFID tag need not be adjacent to an antenna in order for the antenna to receive information from the tag.

RFID technology also has advantages over known methods to monitor dosage amounts during drug delivery. For example, optical technology is imprecise because it relies on visual images to determine the amount administered. Meanwhile, the aforementioned inductive and capacitance methods require close interaction with the syringe in order to determine the volume of a drug within the syringe. This close interaction is invasive and may interfere with the drug delivery process.

The use of radio frequencies for relaying information about the contents of a syringe is already known. U.S. Pat. No. 5,882,338 discloses a medical syringe with one or more data carrier devices that transmit syringe content information (e.g., drug or medicament name, expiration date, concentration and batch number) via radio frequencies. However, U.S. Pat. No. 5,882,338 does not disclose the use of radio frequency transmission to determine the amount of a drug administered from a syringe as the drug is being administered. Indeed, this system requires a syringe pump for the express purpose of using it to closely control the administration of a drug contained in the attached syringe. With the syringe pump controlling the amount of drug administered, there is no need for an external means for monitoring the administration of the drug in the attached syringe.

Additionally, one disadvantage of this system is that in order for the transmission to take place, the information transmitter (i.e., data carrier device) needs to be adjacent or right next to the receiver. This system is specifically designed for a syringe and syringe pump that work in conjunction with one another, in which the syringe fits directly into the syringe pump. The data carrier transmitter is located on the syringe and the receiver is in the syringe pump. This data transmission system is meant to work only when the syringe is connected to the syringe pump. This arrangement includes a disadvantage similar to the situation of using a bar code to identify syringe content information, in that the syringe cannot be in a location remote from a receiver of the information in order for the information to be known.

Accordingly, there is still a need for improvements to drug delivery and monitoring systems, which is fulfilled by the system of the present invention.

### SUMMARY OF THE INVENTION

The present invention relates to the use of RFID technology to allow for closed loop tracking of drugs in a syringe or similar drug delivery device and to determine the volume of the drug within the syringe.

By attaching RFID tags or sensors on a syringe or an attachment to the syringe such as the needle, Luer lock fitting or other devices used to connect the syringe to a receiving device such as an intravenous tubing injection port, information about the contents within the syringe may be transmitted to an antenna or another type of receiver. Additionally, the RFID tags can also be used in a configuration to determine the location of a plunger within a syringe and to track the plunger as it advances within the syringe. In such a configuration, the distance and changes in the distance between a reference point on the plunger and a reference point on the barrel or another non-moving part of the syringe may be determined. Such information allows for the calculation of the volume of a drug in the syringe during administration and allows for the monitoring of the amount of drug administered.

In accordance with various aspects of the present invention, a syringe or a similar drug delivery device is provided one or more tags or sensors. These tags are capable of being programmed with information relating to the contents of the syringe, such as a drug name or another identifier, initial drug volume, drug concentration, expiration date and dispensing technicalities. These tags may also be programmed with a unique identifier (ID) that is associated with an entry in a database which includes the aforementioned information as well as historical information. Such a methodology allows the drug to be tracked with information such as to whom the drug was dispensed, to whom it was delivered, how much was delivered, who returned the drug, and how much wasted. Additionally, the tags are capable of receiving, detecting and/or transmitting radio frequency waves. The tags may be located on separate portions of the syringe that move in relation to another as the drug in the syringe is administered. For example, a plunger and barrel would constitute two such portions on a syringe that move relative to one another as a drug in the syringe is being administered. For the measurement and monitoring of the amount of drug administered, a tag on one portion is an “active” tag while a tag on the other portion is a “passive” tag.

In accordance with one embodiment of the present invention, there is described a drug delivery device comprising first and second portions, in which the portions move relative to one another as a drug contained in the drug delivery device is administered or expelled from the drug delivery device. The drug delivery device includes tags that are mounted on each of the portions. At least one active tag is mounted on at least one of the first or second portions.

In one embodiment, the drug delivery device is a syringe comprising a first portion with a plunger and a second portion. The plunger is movable relative to the second portion such that the plunger is operative for expelling a drug from the syringe.

In another embodiment, an active tag is mounted on one of the first or second portions, and a passive tag is mounted on the other of the first and second portions.

In a particularly preferred embodiment, an active tag is mounted on the plunger and a passive tag is mounted on the second portion.

In yet another embodiment, at least one active tag is mounted on the first portion and at least one active tag is mounted on the second portion.

In accordance with a second embodiment of the present invention, there is described a housing for attaching tags to a drug delivery device. The tags may be active or passive tags.

In one embodiment, the housing may have a tactile, geometric or color configuration, or a combination thereof, for ready identification of the drug housed within the drug delivery device.

In another embodiment, the housing contains an active tag therein, whereby pressure on the housing activates the active tag.

In accordance with another embodiment of the present invention, there is described a method of determining a dose of a drug or medicament administered by a drug delivery device. The method includes providing a drug delivery device including first and second portions that move relative to one another when a drug is being administered. The first and second portions each have corresponding first and second tags mounted to those portions. The first tag is activated to emit a signal with a specified frequency. Once the second tag receives the first tag's signal, the second tag emits a second signal with a different frequency than the first tag's signal. Then, the time delay between the first signal and the second signal is calculated. Based on the time delay between the first and second signals, the movement of the first portion with respect to the second portion is determined and the amount of drug administered is determined.

In one embodiment, for the determination of the time delay, one or more external sensing devices are provided for receipt of the first and second signals. The time delay is calculated as the time elapse between receipt of the first and second signals. Then, the amount of the drug administered is determined based upon the time delay.

In another embodiment, for the calculation of the time delay, a subsystem is provided in the first tag for detection of the second signal. The first tag functions actively and passively. When the second signal reaches the first tag, the first tag receives and detects the second signal. The time delay is calculated as the time elapse between emission of the first signal and receipt of the second signal.

In another embodiment, the detection and calculation of the second signal is performed by a subsystem within the active tag, in which the subsystem is designed to be sensitive to the signal generated by the second tag.

In another embodiment, the sending of the third signal is performed by the subsystem.

In yet another embodiment, if encrypted, the third signal is encrypted by the subsystem.

In accordance with another embodiment of the present invention, there is described a drug identification and delivery monitoring system. The system includes a drug delivery device with first and second portions, in which the portions move relative to one another as a drug contained in the drug delivery device is administered or expelled from the drug delivery device. The drug delivery device includes tags that are mounted on each of the portions. The tags are capable of generating and sending radio frequency signals. The system also includes one or more external sensing devices for receipt and/or detection of signals from the tags.

### BRIEF DESCRIPTION OF THE DRAWINGS

FIG. 1 is a perspective view of a drug delivery device in accordance with certain embodiments of the present invention.

FIG. 2 is a perspective view of a drug delivery device in accordance with FIG. 1, further including a planar flange.

FIG. 3a is a perspective view of a housing for tags in accordance with certain embodiments of the present invention.

FIG. 3b shows various tactile, color or geometric configurations for use with the housing in accordance with FIG. 3a.

FIGS. 4-6 illustrate one operative embodiment of the drug delivery device and drug identification and delivery monitoring system of the present invention.

FIGS. 7a-d illustrate an alternate operative embodiment of the drug delivery device and drug identification and delivery monitoring system of the present invention.

### DETAILED DESCRIPTION

In describing the preferred embodiments of the present invention, specific terminology will be resorted to for the sake of clarity. However, the invention is not intended to be limited to the specific terms so selected, and is to be understood that each specific term includes all technical equivalence which operates in a similar manner to accomplish a similar purpose.

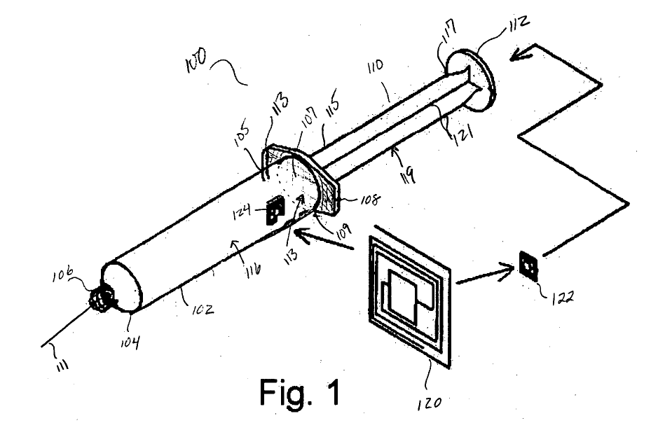

Referring now to the drawings, FIG. 1 depicts a perspective view of a drug delivery device 100 in accordance with one embodiment of the present invention, in the form of a syringe. However, the drug delivery device 100 of the present invention is not limited to syringe form and may be in other forms so long as it includes at least two portions that move relative to one another when a drug contained in the drug delivery device 100 is expelled therefrom or otherwise administered.

Additionally, the drug delivery device 100 may be made from any material. However, relatively non-porous and leak proof materials are preferred. Plastic is one example of a suitable material.

As shown in FIG. 1, the drug delivery device 100 includes two portions 102 and 110. The two portions 102 and 110 move relative to one another when a drug or medicament within is administered or expelled from the drug delivery device 100.

In the particular embodiment shown in FIG. 1, the drug delivery device 100 is in the form of a syringe, and portion 110 is in the nature of a plunger and portion 102 is in the nature of an elongated, hollow, tubular barrel for holding and expelling fluid 116 (e.g., drugs and medicament) therefrom. One such syringe, with a plunger and a barrel, is disclosed in U.S. Pat. No. 5,651,775, the disclosure of which is incorporated herein by reference.

In syringe form, portion 102 of the drug delivery device 100 also includes a tapered end 104 having a protrusion 106 for expelling fluid 116 therefrom, and an opposing end 105 having an opening 107 for insertion of portion 110 into the hollow housing of portion 102. The protrusion 106 may have a needle or a tube 111 attached thereto for administration of fluid 116 to a patient. The opposing end 105 may further include a finger platform 108 extending perpendicularly from the whole, or portion, of the circumference 109 of the opposing end 105. Although the finger platform 108 is shown in a planar form, other forms and shapes are contemplated as well. The finger platform 108 may be made of plastic, rubber, or other rigid material useful for applying pressure thereto. It is also contemplated that the finger platform 108 incorporates a non-slip design 113 for finger placement. Additionally, portion 102 may include readings or marks thereon for optical reading of the amount or kind of fluid 116 housed therein. Preferably, at least the sides of portion 102 are substantially transparent such that measurable light may be transmitted through the sides.

If the drug delivery device 100 is in syringe form, portion 110 is typically as long as the interior of the first portion 102 and fits snugly within the opening 107 created at the opposing end 105 of portion 102. In the embodiment shown in FIG. 1, portion 110 has a first end 115 and a second end 117 and an elongated middle segment 119. In this embodiment, the middle segment 119 is composed of four axially planar flanges 121 connected at right angles to each other. However, other designs for the middle segment 119 are also contemplated. The first end 115 of the portion 110 may include a gasket or seal element 113 attached thereto for creating a liquid-tight seal with the opening 107 of the opposing end 105 of portion 102, while expelling fluid 116 from the drug delivery device 100. Portion 110 may also include a finger pad 112 located on the second end 117, useful for applying pressure to portion 110 to move it within the first portion 102 toward the protrusion 106 and thereby expel a fluid 116.

Also depicted in FIG. 1 are RFID tags or sensors 120 for use in the present invention. As used herein, the terms “RFID tag” and “RFID sensor” are interchangeable and refer to any device capable of receiving, storing and/or sending information utilizing radio frequencies. Aside from the terms “tags” and “sensors,” such devices have also been referred to as “transmitters” and “transponders.” Examples of such RFID devices are discussed in U.S. Pat. Nos. 6,371,375, and 6,496,113, the disclosures of which are herein incorporated by reference. One suitable RFID tag for use in the present invention is the “MM chip,” which is manufactured by Toppan Forms Co, Ltd. These RFID tags can operate at a variety of frequencies from 13.56 MHz to 2.45 GHz, making them useful in a variety of settings. In addition, the “MM chip” can store a 64-bit or 86-bit electronic product code.

The RFID tag for use in the present invention may be “active” or “passive.” Generally, RFID tags are capable of sending and/or receiving data in the form of radio frequency (RF) signals or pulses. As explained above, active tags require an internal power source, and are typically used to emit a pulsing or continuous signal. Suitable internal power sources include built-in batteries and piezoeletronic strips that generate voltage when deformed under pressure, although other power sources are contemplated. Passive tags, on the other hand, do not require a power source within the tag to send a signal. Instead, a passive tag, upon receipt of an outside signal, stores the energy from that signal in a capacitor or any other device capable of storing and releasing an electrical charge, and utilizes that energy to transmit a second signal, which is usually at a different frequency than the outside signal received. For receipt of RF signals, the tags may include one or more antennae, which may be external or internal.

The tags may also include a subsystem capable of detecting specific signals. After such detection, the subsystem may also be configured to generate one or more subsequent signals and transmit the generated signals. Preferably, the subsystem is also capable of encrypting the generated signals prior to their transmission. Additionally, the subsystem may also be configured to perform at least basic mathematical calculations, such as the addition, subtraction, multiplication and division of numbers, and to store the results of the calculations. The subsystem includes any device or hardware, such as an electronic circuit or chip, that is capable of being programmed to detect, generate, transmit and/or encrypt one or more specified signals, with the proviso that the device fits within a tag. In this regard, the subsystem includes at least one processing or computing unit. Preferably, the subsystem also has a memory unit for storage of data. Subsystems that may be adaptable are known as “mote” systems. The subsystem generally perform the calculations to determine the time differentials or may also determine the amount of drug delivered based on differences in the time delay. The subsystem may reside in the ID tag on the syringe.

In one embodiment, the active tag is initially not actively emitting a signal, but is, instead, capable of being turned on or activated by an outside source or stimuli. One such stimulus may be pressure. Accordingly, the active tag may be pressure sensitive and capable of being activated by applying pressure on the tag. Once activated, the active tag's internal power source generates a voltage for emission of a signal.

In the drug delivery device 100 of the present invention, as shown in FIG. 1, at least one active tag 122 is mounted on or within one of two portions 102,110. Optionally, either at least one passive tag 124 or active tag 122 is mounted on or within the other of two portions 102,110. The two portions 102,110 move relative to one another as a drug within the drug delivery device 100 is being administered or expelled therefrom. As the two portions 102,110 move relative to one another, the one or more tags mounted on portion 102 and the one or more tags mounted on portion 110 also move relative to one another, which results in a change in position and distance between the one or more tags mounted on portion 102 and the one or more tags mounted on portion 110. For example, in the particular embodiment shown in FIG. 1, portion 110 is a plunger and portion 102 is a barrel, and one active tag 122 is mounted on the finger pad 112 of portion 110 and one passive tag 124 is mounted on a side of portion 102. To administer or expel the contents within portion 102, pressure is applied to portion 110 so that it moves into portion 110 and toward the protrusion 106. As portion 110 moves into portion 102, the active tag 122 and the passive tag 124 change their positions and move relative to one another, such that the distance between the active tag 122 and passive tag 124 changes as the contents of the drug delivery device 100 are being administered.

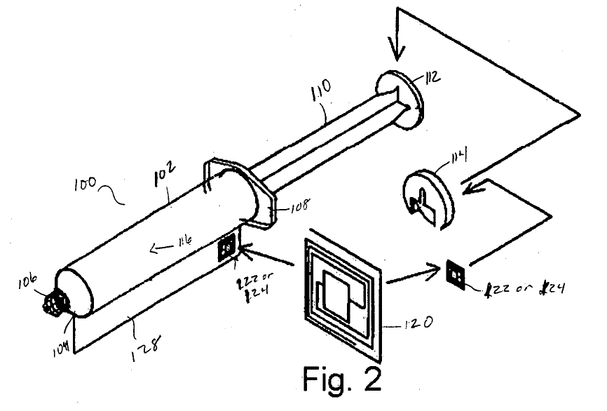

As shown in FIG. 2, in addition to the RFID tags 120 being mounted directly on or within the portions 102,110, the tags 120 may be mounted indirectly on portions 102,110 via extensions, attachments or housings. In this manner, the tags 120 may be mounted on or within such extensions, attachments or housings. The extensions, attachments and housings may be in any form so long as they are engaged or connected to their respective portion 102 or 110. The nature of the engagement or connection is such that the extension, attachment or housing moves in concert with the corresponding portion 102 or 110 to which it is engaged or connected. Additionally, the extension, attachment or housing should not substantially interfere with the administration of the contents of the drug dispensing device 100. Likewise, a portion 102,110 may be adapted to include the aforementioned extensions, attachments or housings so long as these adaptations also do not do not substantially interfere with the administration of the contents of the drug dispensing device 100. For example, if the drug dispensing device is a syringe, tags 120 may be mounted on or within a finger platform 108 or a syringe label cradle.

In the particular embodiment shown in FIG. 2, a tag 120 may be mounted on a flange 128 that protrudes from a portion 102,110. In FIG. 2, the specific portion shown to include the flange 128 is portion 102.

In another particular embodiment, a tag 120 may be mounted in a housing that attaches to a portion 102,110. FIG. 2 shows one embodiment of such a housing, in the form of a heel button 114 that slideably mounts to the finger pad 112 located on the second end 117 of portion 110. Other modes of attaching the heel button 114 to the finger pad 112 are also contemplated, including the use of adhesives.

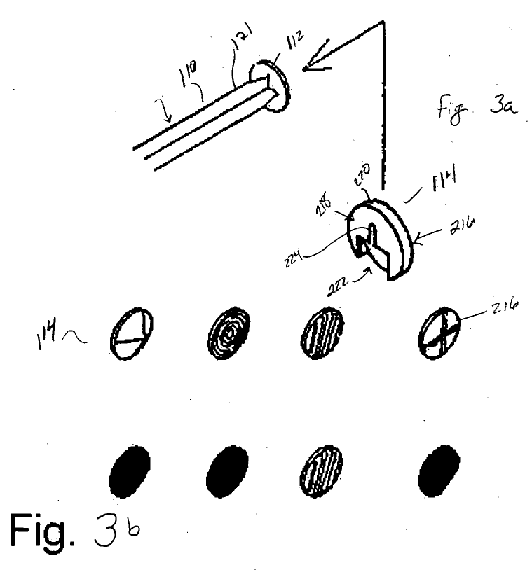

FIG. 3a shows a perspective view of a suitable heel button 114 for use in the present invention. The heel button 114 includes a front face 216 and a back face 218 having a sidewall 220 disposed between the two. The front face 216 is preferably substantially circular, however, other shapes have been contemplated, including, but not limited to, square, triangular, rectangular and spherical shapes. The back face 218 preferably has an opening 222 for mounting onto a finger pad 212 of portion 210. If needed, the back face 218 may also have a notch 224. As described above, in certain embodiments, portion 110 may include one or more axial planar flanges 121. The notch 224 allows one axial planar flange 121 to fit within the notch 224, thereby enabling a secure fit of the heel button 114 to portion 110.

In a particularly embodiment, the front face 216 may also be visually and/or tactilely encoded to identify a fluid 116 housed within the drug delivery device 100, as shown in FIG. 3b. FIG. 3b shows various heel button 114 configurations, but not all possible configurations, as contemplated by this invention. As shown, the heel button 114 may have tactile, color, or geometric configurations in any quantity and combination. Suitable tactile configurations include raised or lowered lines or ridges, circles and bumps, and combinations thereof. Suitable color configurations include one or more colors of any known color (e.g., red, green, blue and yellow), and hues, mixtures and/or combination thereof. Suitable geometric configurations include circles, squares, triangles and ovals, and combinations thereof.

By way of example, to identify the effect of a drug, such as a paralyzing drug, housed within the drug delivery device 100, the heel button 114 may be provided with a sharp central point. Alternatively, the tactile, color, or geometric configurations may also be used to identify the class of drugs housed with in a particular drug delivery device 100. For example, if the fluid 116 housed within the drug delivery device 100 is a narcotic, the heel button 114 may be provided with concentric ridges while parallel bars may be used if the fluid 116 is another class of drugs, such as benzodiazepine.

Such tactile, color, or geometric configurations provide an additional margin of safety in drug administration. One of the most common drug administration errors is picking up a drug delivery device, such as a syringe, that is correctly labeled, but contains an incorrect drug, and administering the incorrect drug to a patient. Another common error is the unintended swapping of one syringe for another. The configurations, such as drug-class specific coloration, reduce drug administration errors, by providing feedback to the administrator of the drugs, which reduces the risks that an unintended drug will be administered. A referral may be made at this point to the creation of specially shaped knobs on anesthesia machines which give feedback to an anesthetist when grasped as to whether the oxygen or nitrous oxide valve is being manipulated.

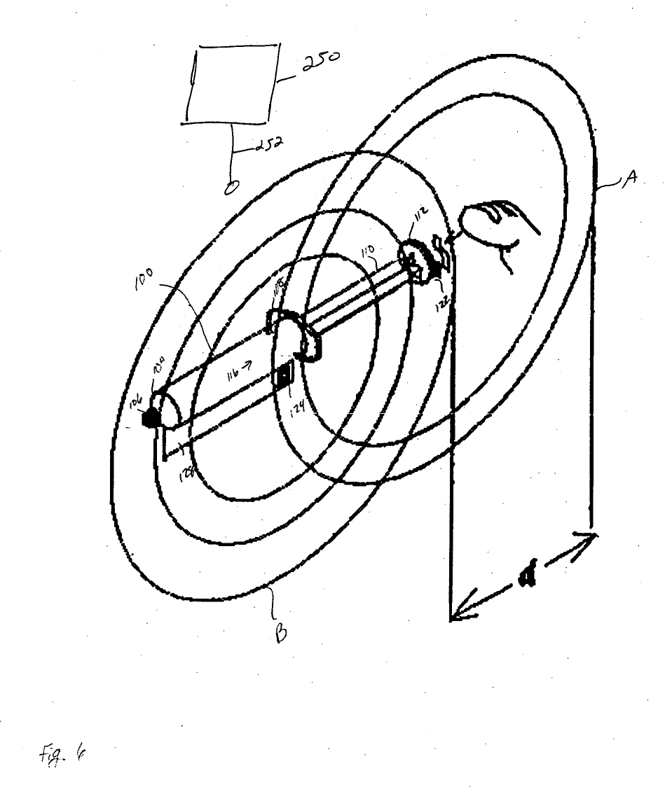

In accordance with another embodiment, the present invention also includes a drug identification and delivery monitoring system. In the system, there is a drug delivery device 100 of the present invention. The drug delivery device 100 includes at least two tags 120. The tags 120 are capable of storing data and communicating this data to and from each other through emission and receipt of signals. Additionally, as shown in FIG. 6, at least one of the tags 120 is capable of sending data to one or more external sensing devices 250. The sensing devices 250 may have one or more antennae 252 for receiving signals. In a preferred embodiment, the external sensing devices 250 are located remotely from the drug delivery device 100 and the tags 120. The communication of signals between the tags 120 and the external sensing devices 250 allow for the monitoring of the identification and amount of an administered drug contained in the drug delivery device 100.

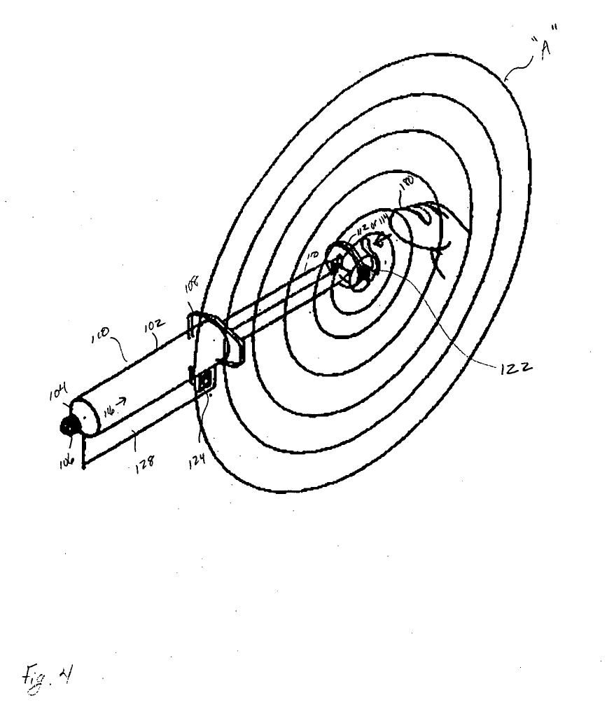

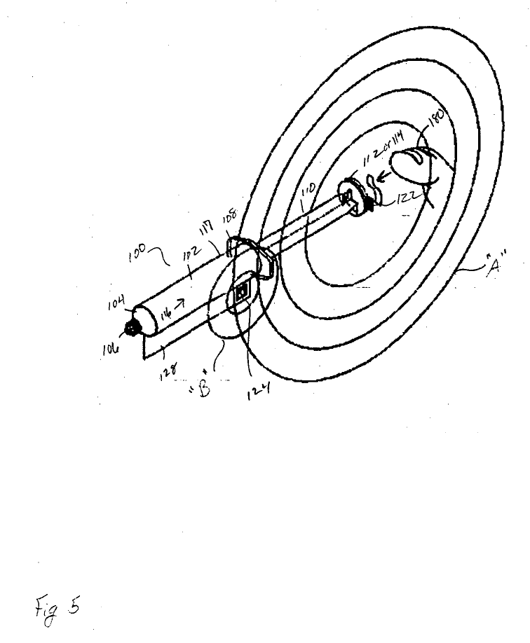

FIGS. 4-6 describe an operative embodiment of the drug delivery device 100 and the drug identification and delivery monitoring system of the present invention. The drug delivery device 100 shown in FIGS. 4-6 includes an active tag 122 mounted on portion 110 and a passive tag 124 mounted on portion 102. As an alternative, the active tag 122 may be mounted on portion 102 and the passive tag 124 mounted on portion 110. The active tag 122 emits a signal (hereinafter “signal A”) at a specific frequency. Once signal A reaches the passive tag 124, the passive tag 124 receives signal A via one or more antennae. As depicted in FIG. 5, in response to receipt of signal A, the passive tag 124 emits its own signal (hereinafter “signal B”) at a different frequency than the frequency of signal A. As shown in FIG. 6, both signals A and B are received by one or more external sensing devices 250. When considered together, the two signals A and B constitute an event that allows for the calculation or determination of the distance between the active tag 122 and the passive tag 124 as their positions change in relation to one another. The time delay or difference, depicted as “d” in FIG. 6, will vary based upon the distance or change in distance between the active tag 122 and passive tag 124. In this instance, the time delay is the time difference between the transmission of signal A and the receipt of signal B.

In the particular embodiment shown in FIGS. 4-6, the drug delivery device 100 is in the form of a syringe, in which portion 110 is in the nature of a plunger and portion 102 is in the nature of an elongated hollow tubular barrel. The active tag 122 is mounted to the finger pad 112 of portion 110 or in a heel button 114 attached to the finger pad 112. As the plunger (or portion 110) is depressed, fluid 116 within the barrel (or portion 116) is expelled. At the same time, the active tag 122 moves closer to the passive tag 124. The active tag 122 may continually emit signals without external stimulation or emit signals with an initial or continued activation, such as from pressure. Alternatively, the active tag 122 may be programmed to begin and/or stop emission of signals at a specified time and/or date. With a decrease in distance between the active tag 122 and the passive tag 124, an emitted signal travels over a smaller distance between the tags 122,124. This shorter distance results in a shorter amount of time required for a signal to travel between the tags 122,124. Therefore, a subsequently emitted signal A reaches the passive tag 126 earlier than the prior signal A that was emitted when the distance between the active tag 122 and the passive tag 124 was greater. In turn, the passive tag 124 emits its signal B earlier as well, which reaches the external sensing device 250 in a shorter amount of time, resulting in a shorter time differential. Accordingly, as the distance between the active tag 122 and the passive tag 124 becomes smaller, the time differential between the receipt of signal A and the receipt of signal B also becomes smaller. This, in effect, allows for the determination of the position, or change in position, of the active tag 122 during drug delivery through remote triangulation of signals A and B.

The triangulation of the data is accomplished using suitable software that calculates the (d) time interval. Although triangulation is preferred to completely accurately determine the relative positions of the two tags, since the degrees of freedom of movement of the plunger is essentially limited to the linear movement of the plunger, the triangulation becomes essentially two dimensional rather than three (thereby simplifying the calculation). This might introduce a small degree of error as the plunger could rotate and therefore the (d) measurement may change solely due to plunger rotation, but the error is anticipated to be small in relation to the range of the plunger movement. In most circumstances the plunger rotates very little as it is being depressed.

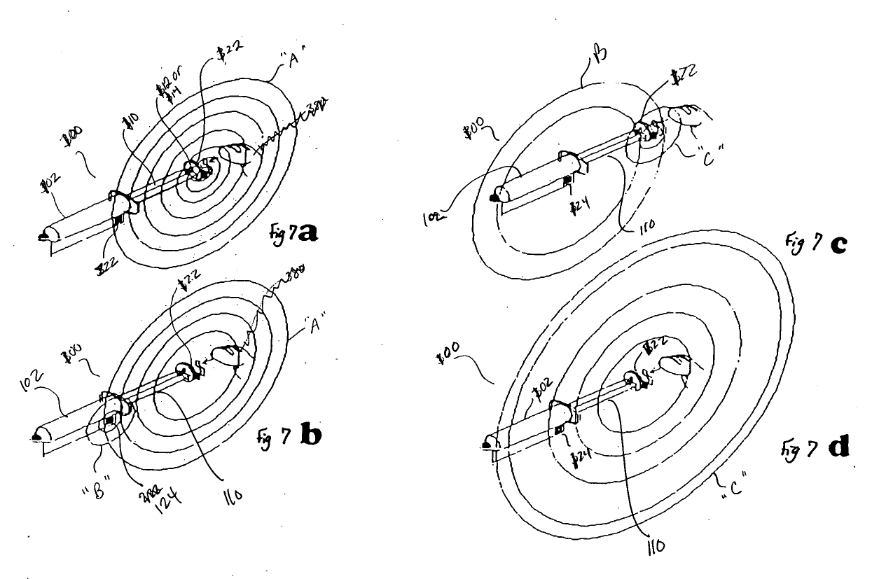

FIGS. 7a-7d describes another operative embodiment of the drug delivery device 100 and the drug identification and delivery monitoring system of the present invention. Similar to the drug delivery device in FIGS. 4-6, the drug delivery device 100 shown in FIGS. 7a-7d also includes an active tag 122 mounted on portion 110 and a passive tag 124 mounted on portion 102. As shown in FIG. 7a, the active tag 122 emits signal A, which has a specific frequency. Once signal A reaches the passive tag 124, the passive tag 124 receives signal A. As depicted in FIG. 7b, in response to receipt of signal A, the passive tag 124 emits its own signal, signal B, at a different frequency than the frequency of signal A. When signal B reaches the active tag 122, the active tag 122 (which also acts passively) receives and detects signal B. The detection may take place through a subsystem (not shown), as described above, in the active tag 122 that is designed to be sensitive to signals generated by the passive tag 124. Then, as shown in FIGS. 7c and 7d, the active tag 122 calculates or determines the time delay between signals A and B, and emits a signal with the time delay data (hereinafter “signal C”) to an external sensing device 250. Signal C is at a different frequency than either of the frequencies of signals A and B. Additionally, signal C may be encrypted before its emission. In this set-up, the subsystem of the active tag 122 may also be configured to perform any of the tasks of calculating the time delay, generating a signal that includes the time delay data, transmitting the signal, and encrypting the signal prior to transmission.

Once received, the external sensing device 250 may then decrypt signal C and/or separate the data containing the time delay information from the rest of the signal, or send the signal to another apparatus for such decryption and/or separation. In this instance, the time delay is the time differential between emission of signal A and detection of signal B by the active tag. This approach eliminates the triangulation requirement for the observing system to be able to detect the distance between the plunger heel and barrel when the syringe is placed at certain angles to the detecting antennae. The first signal from the active tag announces activity with the drug of interest and the second signal repeats the identity but further reports on plunger position of the last signal. This cycle repeats until the active tag stops emitting a signal.

In the particular embodiment shown in FIGS. 7a-d, as in FIGS. 4-6, the drug delivery device 100 is in the form of a syringe, in which portion 110 is in the nature of a plunger and portion 102 is in the nature of an elongated hollow tubular barrel. The active tag 122 is mounted to the finger pad 112 of portion 110 or in a heel button 114 attached to the finger pad 112. As the plunger (or portion 110) is depressed), fluid 116 within the barrel (or portion 116) is expelled. At the same time, the active tag 122 moves closer to the passive tag 124. The active tag 122 may continually emit signals without external stimulation or emit signals with an initial or continued activation, such as from pressure. Alternatively, the active tag 122 may be programmed to begin and/or stop emission of signals at a specified time and/or date. With a decrease in distance between the active tag 122 and the passive tag 124, an emitted signal travels over a smaller distance between the tags 122,124. This shorter distance results in a shorter amount of time required for a signal to travel between the tags 122,124. Therefore, a subsequently emitted signal A reaches the passive tag 126 earlier than the prior signal A that was emitted when the distance between the active tag 122 and the passive tag 124 was greater. In turn, the passive tag 124 emits its signal B earlier as well, which when coupled with the narrowing distance between the active tag 122 and the passive tag 124 results in signal B reaching the active tag 122 earlier than the signal B that was emitted when the distance between the active tag 122 and the passive tag 124 was greater. Accordingly, as the distance between the active tag 122 and the passive tag 124 becomes smaller, the time differential between the emission of signal A and the detection of signal B also becomes smaller. The change in time differential determines the position of the active tag 122, or its change in position, as a drug is administered.

In the foregoing operative embodiments, the cycle of signal emissions and receipt and/or detection of those signals may be repeated as the contents in the drug delivery device 100 are administered. The repeated cycle allows for the monitoring of the change in position of portion 102 in relation to portion 110 (or passive tag 124 in relation to active tag 122) as a drug or medicament in the drug delivery device 100 is administered. Since such a change in position directly relates to and is an indication of the change in volume of the drug or medicament within the drug delivery device 100, the repeated cycle allows for the monitoring of an amount of a drug administered as well as the identification of the drug being administered.

Additionally, any of signals A, B or C may contain data relaying syringe content information (e.g., drug or medicament name, expiration date, concentration and batch number), or other data such as syringe size, patient information, billing information, dispensing technique, etc. This data will be received by an external source which can store and/or record the data as may be required. Meanwhile, signal C may also contain data including time delay information, as described above.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A drug delivery device for the administration of a drug, said device comprising:
a first portion and a second portion, said first and second portions being moveable relative to one another when a drug contained in said drug delivery device is being administered;
at least one first tag mounted to said first portion, said first tag adapted to at least transmit first signals; and
at least one second tag mounted to said second portion, said second tag being adapted to receive said first signals and to transmit second signals.

2. The drug delivery device of claim 1, wherein said second tag is a passive tag.

3. The drug delivery device of claim 1, wherein said first tag is an active tag.

4. The drug delivery device of claim 1, wherein at least one of said first and second tags store and transmit drug identification information.

5. The drug delivery device of claim 4, wherein said information is said drug's name, expiration date, NDC number, unique identification number, national drug identification code or a unique tracking code, concentration, batch number, or a combination thereof.

6. The drug delivery device of claim 5, wherein said information is included in one of said signals.

7. The drug delivery device of claim 1, wherein said first and second signals have different frequencies.

8. The drug delivery device of claim 1, wherein said first tag is pressure activated.

9. The drug delivery device of claim 1, wherein said first tag includes an internal power source.

10. The drug delivery device of claim 9, wherein said power source is a battery, inductively charged, photovoltaic or a piezoelectric element.

11. The drug delivery device of claim 1, wherein said first tag includes a subsystem and said first tag is adapted to receive said second signals.

12. The drug delivery device of claim 11, wherein said subsystem is adapted to calculate a time delay between transmission of at least one of said first signals and receipt thereby of at least one of said second signals.

13. The drug delivery device of claim 1, further comprising an attachment for securing one of said first and second tags to one of said first and second portions.

14. The drug delivery device of claim 13, wherein said attachment is a heel button.

15. The drug delivery device of claim 14, wherein said attachment includes at least one tactile, geometric or color configuration.

16. The drug delivery device of claim 1, wherein said first and second signals comprise radio frequency signals.

17. The drug delivery device of claim 1, wherein said drug delivery device is a syringe and said first portion is a plunger that is operative for expelling said drug from said syringe.

18. The drug delivery device of claim 17, wherein said second portion comprises a barrel of said syringe.

19. A drug identification and delivery monitoring system comprising:
a drug delivery device including a first portion and a second portion, said first and second portions being moveable relative to one another when a drug contained in said drug delivery device is being administered;
at least one first tag mounted to said first portion, said first tag being adapted to at least transmit first signals;
at least one second tag mounted to said second portion, said second tag adapted to receive said first signals and to transmit second signals; and
at least one sensing device adapted to receive said first and second signals for determining the amount of said drug being administered;
wherein at least one of said first and second tags stores and transmits drug identification information.

20. A method for determining a volume of a drug being administered during drug delivery comprising:
providing a drug delivery device including first and second portions moveable relative to one another when a drug contained in said drug delivery device is being administered, said drug delivery device having a first tag mounted to one of said first and second portions and a second tag mounted to the other of said first and second portions;
transmitting a first signal from said first tag;
transmitting a second signal from said second tag responsive to said first signal;
calculating a time delay between said first and second signals; and
determining the volume of said drug administered based on said time delay.

21. The method of claim 20, wherein said first tag is an active tag and said second tag is a passive tag.

22. The method of claim 21, second signal is transmitted after receipt of said first signal from said active tag by said passive tag.

23. The method of claim 20, wherein both first and second signals are radio frequency signals.

24. The method of claim 20, wherein one of said first and second signals include drug identification information.

25. The method of claim 24, wherein said information is said drug's name, expiration date, concentration or batch number, NDC number, unique identification number, or a combination thereof.

26. The method of claim 20, further including activating said first tag by applying pressure thereto.

27. The method of claims 20, wherein said first and second signals have different frequencies.

28. The method of claim 20, further comprising providing at least one external sensing device for receiving said first and second signals.

29. The method of claim 28, wherein said time delay is the time differential between said receipt of said first signal and said receipt of said second signal.

30. A method of determining a volume of a drug being administered during drug delivery comprising:
providing a drug delivery device including first and second portions moveable relative to one another when a drug contained in said drug delivery device is administered, said drug delivery device having an active tag mounted to one of said first and second portions and a passive tag mounted to the other of said first and second portions;
transmitting a first signal from said active tag;
transmitting a second signal from said passive tag after receipt of said first signal by said passive tag;
calculating a time delay between said first and second signals after receipt of said second signal by said active tag;
transmitting a third signal including time delay information by said active tag; and
determining a volume of said drug administered based on said time delay.

31. The method of claim 31, wherein said time delay is the time differential between said transmission of said first signal and said detection of said second signal.

32. The method of claim 31, wherein said third signal is encrypted prior to its transmission.

33. The method of claim 31, wherein said third signal includes drug identification information.

34. The method of claim 34, wherein said drug identification information is said drug's name, expiration date, concentration, batch number, unique identification number or a combination thereof.

35. The method of claim 31, further providing an external sensing device for receipt of said third signal.

36. The method of claim 31, wherein said active tag includes a subsystem for detecting said second signal.

37. The method of claim 31, wherein said first, second and third signals comprise radio frequency signals.

38. The method of claim 37, wherein said first, second and third signals are each at a different frequency.
